(19) United States
(12) Patent Application Publication
(10) Pub. No.: US 2026/0017541 A1
(43) Pub. Date: Jan. 15, 2026

(54) INFORMATION PROCESSING APPARATUS, METHOD, AND NON-TRANSITORY COMPUTER-READABLE MEDIUM FOR TASK INFERENCE USING MACHINE LEARNING WITH GRADIENT BASED MULTI-TASK LEARNING

(71) Applicant: NEC Corporation, Tokyo (JP)

(72) Inventor: Takuro SHIMAYA, Tokyo (JP)

(73) Assignee: NEC Corporation, Tokyo (JP)

(21) Appl. No.: 19/259,112

(22) Filed: Jul. 3, 2025

(30) Foreign Application Priority Data
Jul. 12, 2024 (JP) 2024-112780

(51) Int. Cl. G06N 5/04; G16H 30/40; G16H 50/20

(52) U.S. Cl. CPC G06N 5/04; G16H 30/40; G16H 50/20

(57) ABSTRACT

An information processing apparatus includes at least one memory storing instructions, and at least one processor configured to execute the instructions to acquire data to be input to a first layer included in a plurality of layers forming a learned model generated by multi-task learning, acquire an inference result of a subtask, the inference result being output from a second layer, which is a layer subsequent to the first layer, by inputting the data to the first layer, acquire a subtask label corresponding to the data, calculate a gradient in the data of a function using, as inputs, the inference result of the subtask and the subtask label, and perform task inference using the data, the gradient, and the learned model. As an example, the information processing apparatus is used for decision-making assistance such as diagnosis using a medical image.

### INCORPORATION BY REFERENCE

This application is based upon and claims the benefit of priority from Japanese patent application No. 2024-112780, filed on Jul. 12, 2024, the disclosure of which is incorporated herein in its entirety by reference.

### TECHNICAL FIELD

The present disclosure relates to an information processing apparatus, an information processing method, and a program.

### BACKGROUND ART

A method of learning a machine learning model by multi-task learning has been proposed. The multi-task learning is a method of improving accuracy by causing one model to simultaneously perform learning for a plurality of tasks related to a target task. For example, JP 2021-174428 A describes detecting a symptom of a disease or a sign of severity using a learned model constructed by a machine learning algorithm such as multi-task learning.

### SUMMARY

In a case where inference is performed using a learned model generated by multi-task learning, it is conceivable that accuracy of task inference is improved by a user changing an inference result of a subtask. Subtask can also be referred to as “auxiliary task”. The technique described in JP 2021-174428 A has a problem that the effect of change by the user cannot be reflected in the inference of other subtasks.

The present disclosure has been made in view of the above-described problems, and an example object thereof is to provide a technique for performing task inference with higher accuracy using a learned model generated by multi-task learning.

An information processing apparatus according to an example aspect of the present disclosure includes at least one memory storing instructions, and at least one processor configured to execute the instructions to; acquire data to be input to a first layer included in a plurality of layers forming a learned model generated by multi-task learning, acquire an inference result of a subtask, the inference result being output from a second layer, which is a layer subsequent to the first layer, by inputting the data to the first layer, acquire a subtask label corresponding to the data, calculate a gradient in the data of a function using, as inputs, the inference result of the subtask and the subtask label, and perform task inference using the data, the gradient, and the learned model.

An information processing method according to an example aspect of the present disclosure includes data acquisition processing of acquiring, by at least one processor, data to be input to a first layer included in a plurality of layers forming a learned model generated by multi-task learning, inference result acquisition processing of acquiring, by the at least one processor, an inference result of a subtask, the inference result being output from a second layer, which is a layer subsequent to the first layer, by inputting the data to the first layer, subtask label acquisition processing of acquiring, by the at least one processor, a subtask label corresponding to the data, gradient calculation processing of calculating, by the at least one processor, a gradient in the data of a function using, as inputs, the inference result of the subtask and the subtask label, and inference processing of performing, by the at least one processor, task inference using the data, the gradient, and the learned model.

A non-transitory computer-readable medium according to an example aspect of the present disclosure stores a program that causes a computer to execute a data acquisition process of acquiring data to be input to a first layer included in a plurality of layers forming a learned model generated by multi-task learning, an inference result acquisition process of acquiring an inference result of a subtask, the inference result being output from a second layer, which is a layer subsequent to the first layer, by inputting the data to the first layer, a subtask label acquisition process of acquiring a subtask label corresponding to the data, a gradient calculation process of calculating a gradient in the data of a function using, as inputs, the inference result of the subtask and the subtask label, and an inference process of performing task inference using the data, the gradient, and the learned model.

According to an example aspect of the present disclosure, there is an exemplary effect that it is possible to provide a technique for performing task inference with higher accuracy using a learned model generated by multi-task learning.

### BRIEF DESCRIPTION OF DRAWINGS

The above and other aspects, features, and advantages of the present disclosure will become more apparent from the following description of certain example embodiments when taken in conjunction with the accompanying drawings, in which:

FIG. 1 is a block diagram illustrating a configuration of an information processing apparatus according to the present disclosure;

FIG. 2 is a flowchart illustrating a flow of an information processing method according to the present disclosure;

FIG. 3 is a block diagram illustrating a configuration of an information processing apparatus according to the present disclosure;

FIG. 4 is a diagram illustrating an example of a configuration of a learned model according to the present disclosure;

FIG. 5 is a flowchart illustrating an example of the flow of the information processing method according to the present disclosure;

FIG. 6 is a diagram illustrating an example of a flow of task inference using the information processing apparatus according to the present disclosure;

FIG. 7 is a diagram illustrating an example of change information output by an output control unit according to the present disclosure; and

FIG. 8 is a block diagram illustrating a configuration of a computer functioning as the information processing apparatus according to the present disclosure.

### EXAMPLE EMBODIMENT

Hereinafter, example embodiments of the present disclosure will be exemplified. However, the present disclosure is not limited to the example embodiments described below, and various modifications can be made within the scope described in the claims. For example, example embodiments obtained by appropriately combining techniques (some or all of things or methods) adopted in the following example embodiments can also be included in the scope of the present disclosure. In addition, example embodiments obtained by appropriately omitting some of the techniques adopted in the following example embodiments can also be included in the scope of the present disclosure. In addition, effects mentioned in the following example embodiments are examples of effects expected in the example embodiments, and do not define the extension of the present disclosure. That is, example embodiments that do not achieve the effects mentioned in the following example embodiments can also be included in the scope of the present disclosure.

### First Example Embodiment

A first example embodiment, which is an example of an example embodiment of the present disclosure, will be described in detail with reference to the drawings. The present example embodiment is a basic form of each example embodiment described below. Note that an application range of each technique adopted in the present example embodiment is not limited to the present example embodiment. That is, each technique adopted in the present example embodiment can also be adopted in the other example embodiments included in the present disclosure within a range in which no particular technical problem occurs. In addition, each technique illustrated in the drawings referred to for description of the present example embodiment can also be employed in the other example embodiments included in the present disclosure within a range in which no particular technical problem occurs.

### (Configuration of Information Processing Apparatus)

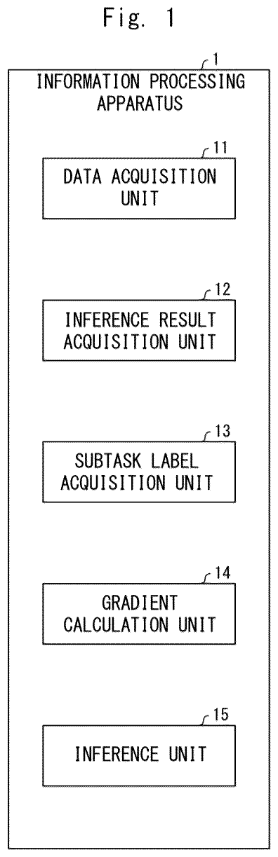

A configuration of an information processing apparatus 1 will be described with reference to FIG. 1. FIG. 1 is a block diagram illustrating a configuration of the information processing apparatus 1. As illustrated in FIG. 1, the information processing apparatus 1 includes a data acquisition unit 11, an inference result acquisition unit 12, a subtask label acquisition unit 13, a gradient calculation unit 14, and an inference unit 15.

The data acquisition unit 11 acquires data to be input to a first layer included in a plurality of layers constituting a learned model generated by multi-task learning. The inference result acquisition unit 12 acquires an inference result of a subtask, which is output from a second layer which is a layer subsequent to the first layer by inputting data to the first layer. The subtask label acquisition unit 13 acquires a subtask label corresponding to data. The gradient calculation unit 14 calculates a gradient in the data of a function having the inference result of the subtask and the subtask label as inputs. As an example, the function is a function representing an error between the inference result of the subtask and the subtask label, but is not limited thereto. As an example, the function may be a function representing a correlation between the inference result of the subtask and the subtask label. The inference unit 15 performs task inference using the data, the gradient, and the learned model.

### (Effects of Information Processing Apparatus)

As described above, the information processing apparatus 1 adopts a configuration including the data acquisition unit 11 that acquires data to be input to a first layer included in a plurality of layers constituting a learned model generated by multi-task learning, the inference result acquisition unit 12 that acquires an inference result of a subtask, which is output from a second layer that is a layer subsequent to the first layer by inputting the data to the first layer, the subtask label acquisition unit 13 that acquires a subtask label corresponding to data, the gradient calculation unit 14 that calculates a gradient in the data of a function having the inference result of the subtask and the subtask label as inputs, and the inference unit 15 that performs task inference using the data, the gradient, and the learned model. Therefore, according to the information processing apparatus 1, it is possible to obtain an effect that task inference can be performed more accurately using a learned model generated by multi-task learning.

### (Flow of Information Processing Method)

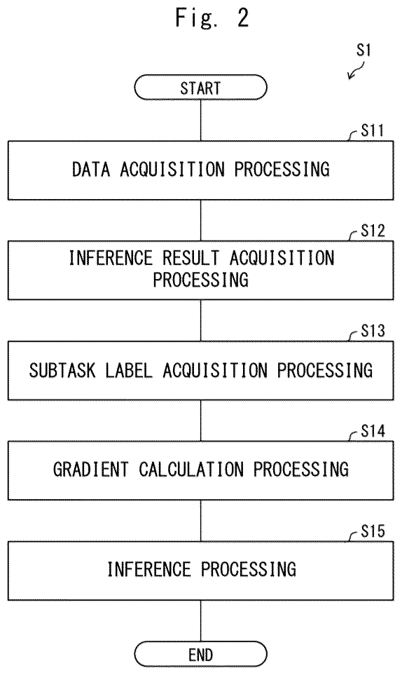

A flow of an information processing method S1 will be described with reference to FIG. 2. FIG. 2 is a flowchart illustrating the flow of the information processing method S1. As illustrated in FIG. 2, the information processing method S1 includes data acquisition processing S11, inference result acquisition processing S12, subtask label acquisition processing S13, gradient calculation processing S14, and inference processing S15.

In the data acquisition processing S11, at least one processor acquires data to be input to a first layer included in a plurality of layers constituting a learned model generated by multi-task learning. In the inference result acquisition processing S12, the at least one processor acquires an inference result of a subtask, which is output from a second layer which is a layer subsequent to the first layer by inputting the data to the first layer. In the subtask label acquisition processing S13, the at least one processor acquires a subtask label corresponding to the data. In the gradient calculation processing S14, the at least one processor calculates a gradient in the data of a function using, as inputs, the inference result of the subtask and the subtask label. In the inference processing S15, the at least one processor performs task inference using the data, the gradient, and the learned model.

### (Effect of Information Processing Method)

As described above, the information processing method S1 adopts a configuration including the data acquisition processing S11 of acquiring, by the at least one processor, data to be input to a first layer included in a plurality of layers constituting a learned model generated by multi-task learning, the inference result acquisition processing S12 of acquiring, by the at least one processor, an inference result of a subtask, which is output from a second layer that is a layer subsequent to the first layer by inputting the data to the first layer, the subtask label acquisition processing S13 of acquiring, by the at least one processor, a subtask label corresponding to the data, the gradient calculation processing S14 of calculating, by the at least one processor, a gradient in the data of a function using the inference result of the subtask and the subtask label as inputs, and the inference processing S15 of inferring, by the at least one processor, a task using the data, the gradient, and the learned model. Therefore, according to the information processing method S1, it is possible to obtain an effect that task inference can be performed with higher accuracy using a learned model generated by multi-task learning.

### Second Example Embodiment

A second example embodiment, which is an example of an example embodiment of the present disclosure, will be described in detail with reference to the drawings. Components having the same functions as the components described in the above-described example embodiment will be denoted by the same reference numerals, and the description thereof will be appropriately omitted. Note that an application range of each technique adopted in the present example embodiment is not limited to the present example embodiment. That is, each technique adopted in the present example embodiment can also be adopted in the other example embodiments included in the present disclosure within a range in which no particular technical problem occurs. In addition, each technique illustrated in each of the drawings referred to for description of the present example embodiment can be employed in the other example embodiments included in the present disclosure within a range in which no particular technical problem occurs.

### (Configuration of Information Processing Apparatus)

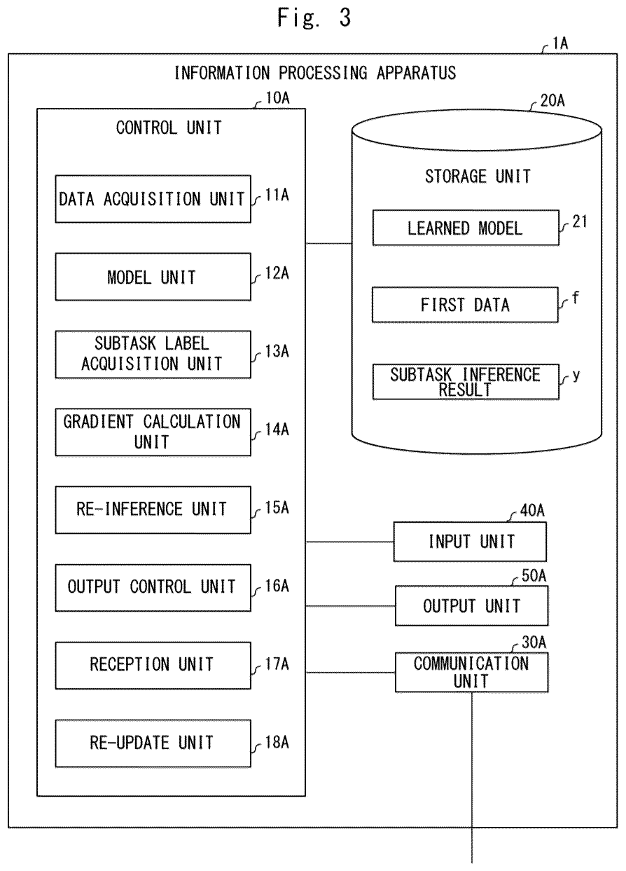

Next, a configuration of an information processing apparatus 1A will be described with reference to FIG. 3. The information processing apparatus 1A is an apparatus that performs task inference using a learned model generated by multi-task learning. Examples of the task include an image recognition task. More specifically, as an example, the information processing apparatus 1A is used for decision-making assistance such as diagnosis using a medical image.

FIG. 3 is a block diagram illustrating the configuration of the information processing apparatus 1A. The information processing apparatus 1A includes a control unit 10A, a storage unit 20A, a communication unit 30A, an input unit 40A, and an output unit 50A.

### (Communication Unit)

The communication unit 30A communicates with a device outside the information processing apparatus 1A via a communication line. Although a specific configuration of the communication line is not limited to the present example embodiment, the communication line is, for example, a wireless local area network (LAN), a wired LAN, a wide area network (WAN), a public line network, a mobile data communication network, or a combination thereof. The communication unit 30A transmits data supplied from the control unit 10A to another device, and supplies data received from another device to the control unit 10A.

### (Input Unit)

The input unit 40A is a configuration for receiving an input to the information processing apparatus 1A, and includes, as an example, an input device such as a keyboard, a mouse, a touch panel, a camera, or a microphone. Furthermore, the input unit 40A may be configured to receive data from the input device via, for example, an interface such as a universal serial bus (USB).

### (Output Unit)

The output unit 50A is a configuration for performing output from the information processing apparatus 1A, and includes, as an example, an output device such as a display, a printer, a touch panel, or a speaker. The output unit 50A may include, for example, an interface such as a USB, and may be configured to output data to the output device via the interface.

### (Storage Unit)

The storage unit 20A stores various pieces of data referred to by the control unit 10A. In particular, the storage unit 20A stores a command of a computer program executed by the control unit 10A. In addition, examples of the data stored in the storage unit 20A include a learned model 21, first data f, and a subtask inference result y. Here, storing the learned model 21 in the storage unit 20A means that a parameter defining the learned model 21 is stored in the storage unit 20A. The first data f and the subtask inference result y are data referred to by a gradient calculation unit 14A to be described later. Details of the first data f and the subtask inference result y will be described later.

### (Learned Model)

The learned model 21 is a machine learning model generated by multi-task learning. The learned model 21 has a plurality of layers. The plurality of layers include, for example, an input layer, a plurality of intermediate layers, and an output layer. The input data to be input to the learned model 21 is, for example, text data, voice data, image data, or a combination thereof. More specifically, the input data is, for example, data representing a medical image. Examples of the medical image include, but are not limited to, an X-ray image, an endoscopic image, a pathological image, an MRI image, or a CT image.

The output of the learned model 21 is an inference result of a main task, and is, for example, a differentiation result regarding a medical image. As an example, the differentiation result includes, but is not limited to, data indicating a disease name and a symptom. Furthermore, the learned model 21 may output a plurality of findings regarding the medical image as the inference result of the subtask. Examples of the inference result of the subtask include, but are not limited to, a shape finding (“circular” and the like), a position finding (coordinate information and the like), and the like.

### (Control Unit)

The control unit 10A includes a data acquisition unit 11A, a model unit 12A, a subtask label acquisition unit 13A, the gradient calculation unit 14A, a re-inference unit 15A, an output control unit 16A, a reception unit 17A, and a re-update unit 18A. Each unit of the control unit 10A is enabled by the control unit 10A reading and executing the command of the computer program stored in the storage unit 20A. The model unit 12A is an example of a data acquisition means and an inference result acquisition means according to the present disclosure. The subtask label acquisition unit 13A is an example of a subtask label acquisition means according to the present disclosure. The gradient calculation unit 14A is an example of a gradient calculation means according to the present disclosure. The re-inference unit 15A is an example of an inference means according to the present disclosure. The output control unit 16A is an example of an output control means according to the present disclosure. The reception unit 17A is an example of a reception means according to the present disclosure. The re-update unit 18A is an example of a re-update means according to the present disclosure.

### (Data Acquisition Unit)

The data acquisition unit 11A acquires input data to be input to the learned model 21. As an example, the data acquisition unit 11A may acquire the input data by receiving the input data from another device connected via the communication unit 30A. In addition, the data acquisition unit 11A may acquire input data input to the input unit 40A. Furthermore, the data acquisition unit 11A may acquire the input data by reading the input data from a storage destination (a storage device in the information processing apparatus 1A or a storage device outside the information processing apparatus 1A may be used) designated by a user of the information processing apparatus 1A.

### (Model Unit)

The model unit 12A performs multi-task inference by inputting input data to the learned model 21. In addition, the model unit 12A acquires the first data f input to the first layer included in the plurality of layers constituting the learned model 21, and acquires the subtask inference result y output from the second layer by inputting the first data f to the first layer. The first data f may be input data input to the input layer of the learned model 21, or may be an intermediate feature extracted from the input data. In other words, the first data f may include at least one of input data of the learned model 21 and a feature extracted from the input data.

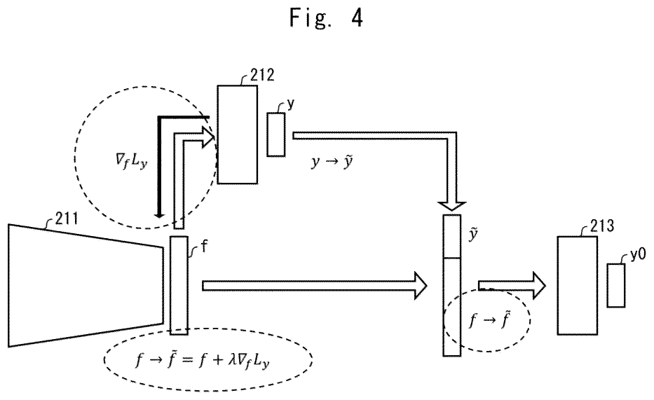

FIG. 4 is a diagram illustrating an example of a configuration of the learned model 21 to which the model unit 12A inputs input data. In the example of FIG. 4, the learned model 21 includes an encoder 211, a first linear layer 212, and a second linear layer 213. The encoder 211, the first linear layer 212, and the second linear layer 213 each include a plurality of layers. Therefore, the encoder 211, the first linear layer 212, and the second linear layer 213 can also be referred to as “the encoder block”, “the first block”, and “the second block”, respectively, where “the block” means the block of layers.

The model unit 12A inputs input data to the encoder 211. The encoder 211 outputs the first data f which is an intermediate feature. In the example of FIG. 4, the first data f is, for example, an n-dimensional (n is a natural number) vector. In addition, the model unit 12A inputs the first data f, which is the intermediate feature, to the first linear layer 212, and the first linear layer 212 outputs a subtask inference result y, which is an inference result of the subtask. The subtask inference result y may be, for example, binary data indicating an inference result, or may be a real number indicating an inference result.

In addition, the model unit 12A inputs, to the second linear layer 213, data obtained by combining the first data f, which is the intermediate feature, and the subtask inference result y. The second linear layer 213 outputs an inference result y0 of the task. The inference result y0 may be, for example, binary data indicating an inference result or a real number indicating an inference result.

### (Subtask Label Acquisition Unit)

The subtask label acquisition unit 13A acquires a subtask label ^{˜}y. The subtask label ^{˜}y is a label of a subtask inferred by the learned model 21, and is, for example, a class classification label or a bounding box, but is not limited thereto. More specifically, the subtask label ^{˜}y may be, for example, a label indicating an inference result of a disease state regarding a medical image. Here, as an example, the inference result may include findings (findings of the shape of a tissue, findings of the anatomical position, and the like) that can be diagnosed from the image. The number of subtask labels ^{˜}y acquired by the subtask label acquisition unit may be one or more. The expression “^{˜}y” represents “y with tilde”.

As an example, the subtask label acquisition unit 13A may acquire the subtask label ^{˜}y by receiving the subtask label ^{˜}y from another apparatus (a user terminal and the like) connected via the communication unit 30A. Further, the subtask label acquisition unit 13A may acquire the subtask label ^{˜}y input to the input unit 40A. Furthermore, the subtask label acquisition unit 13A may acquire the subtask label ^{˜}y by reading the subtask label ^{˜}y from a storage destination (a storage device in the information processing apparatus 1A or a storage device outside the information processing apparatus 1A may be used) designated by the user of the information processing apparatus 1A.

As an example, the subtask label ^{˜}y is data input by the user of the information processing apparatus 1A using an input device such as a mouse or a keyboard. Examples of the user of the information processing apparatus 1A include a medical worker. In a case where the user inputs the subtask label ^{˜}y, the subtask label acquisition unit 13A may cause a display device to display a pull-down list for selecting a subclass and acquire a subclass label ^{˜}y selected from the pull-down list by the user, as an example. In addition, for example, the subtask label acquisition unit 13A may display a seek bar for designating a reliability score on the display device, and acquire the reliability score input by the user operating the seek bar as the subtask label ^{˜}y. Furthermore, for example, the subtask label acquisition unit 13A may cause the display device to display a screen for adjustment of a bounding box, and acquire the bounding box corrected by the user using the input device as the subtask label ^{˜}y.

Further, as an example, the subtask label acquisition unit 13A may acquire the subtask label ^{˜}y by reading the subtask label ^{˜}y from a predetermined database. More specifically, for example, in a case where the subtask is a genotypic inference task, the subtask label acquisition unit 13A may acquire a genetic test result by a blood test from an electronic medical record. Furthermore, the subtask label acquisition unit 13A may acquire, for example, information such as age, gender, race, and the like from a predetermined database as the subtask label ^{˜}y.

### (Gradient Calculation Unit)

The gradient calculation unit 14A calculates a gradient ∇_{f}L_{y} in the first data f of an error between the subtask inference result y and the subtask label ^{˜}y. Here, the gradient ∇_{f}L_{y} is obtained by differentiating an error function L_{y} with the first data f.

### (Re-Inference Unit)

The re-inference unit 15A performs task inference using the first data f, the gradient ∇_{f}L_{y}, and the learned model 21. As an example, the re-inference unit 15A updates the first data f using the gradient ∇_{f}L_{y}, and performs inference using the updated data ^{˜}f obtained by the update and the learned model 21. The updated data ^{˜}f is expressed by the following equation as an example. In the following equation, λ represents a scalar constant.

$${∼}^{} f = f + λ {∇}_{f} {L}_{y}$$

In other words, the re-inference unit 15A back-propagates the gradient ∇_{f}L_{y} to the first data f and updates the first data f using the gradient ∇_{f}L_{y}. Furthermore, the re-inference unit 15A further infers another subtask from the updated data ^{˜}f by forward propagation again. More specifically, as an example, the re-inference unit 15A inputs the updated data ^{˜}f to a third layer included in the plurality of layers constituting the learned model 21 in the multi-task inference. In the example of FIG. 4, the re-inference unit 15A inputs data obtained by combining the updated data ^{˜}f and the subtask inference result ^{˜}y to the second linear layer 213.

### (Output Control Unit)

The output control unit 16A outputs information indicating a multi-task inference result. In addition, the output control unit 16A outputs update information indicating a content of the update by the re-inference unit 15A. As an example, the output control unit 16A may output the information by writing the information in a storage destination (a storage device in the information processing apparatus 1A or a storage device outside the information processing apparatus 1A may be used) designated by the user of the information processing apparatus 1A. Furthermore, the output control unit 16A may transmit the information via the communication unit 30A, or may output the information to an output device such as a display.

The information indicating the multi-task inference result output by the output control unit 16A may be, for example, information indicating an inference result of a disease state regarding a medical image. More specifically, the information indicating the inference result may be malignancy (“0.3”, “0.8”, and the like) as an example.

The update information output by the output control unit 16A may be, for example, the updated data ^{˜}f, or may be information indicating a difference between the updated data ^{˜}f and the first data f before update. Furthermore, as an example, the update information may be information indicating how much the updated data ^{˜}f has changed from the data before the update or information indicating how the updated data ^{˜}f has changed from the data before the update. In a case where the updated data ^{˜}f is a text, the output control unit 16A may visualize and output a word related to the change using, for example, an attention map. Furthermore, in a case where the updated data ^{˜}f is image data, the output control unit 16A may display the data before update and the data after update side by side on a display device, or may display an image representing a difference between the data before update and the data after update on the display device.

### (Reception Unit)

The reception unit 17A receives change instruction information instructing a change to the updated data ^{˜}f. As an example, the reception unit 17A receives the change instruction information input to the input unit 40A by a user operation or the like. The change instruction information is, for example, information in which the user designates a portion that should not be changed in the update information output by the output control unit 16A. For example, in a case where a portion not related to the subtask is changed in the updated data ^{˜}f, the user performs an operation such that the portion is not changed, and the reception unit 17A receives the change instruction information based on the user operation.

### (Re-Update Unit)

The re-update unit 18A updates the updated data ^{˜}f again using the change instruction information received by the reception unit 17A. As an example, the re-update unit 18A performs processing of returning a portion indicated by the change instruction information to the original position in the updated data ^{˜}f updated by the re-inference unit 15A. The re-inference unit 15A performs multi-task inference by using re-update data ^{˜˜}f obtained by allowing the re-update unit 18A to update the updated data ^{˜}f.

### (Flow of Inference Method)

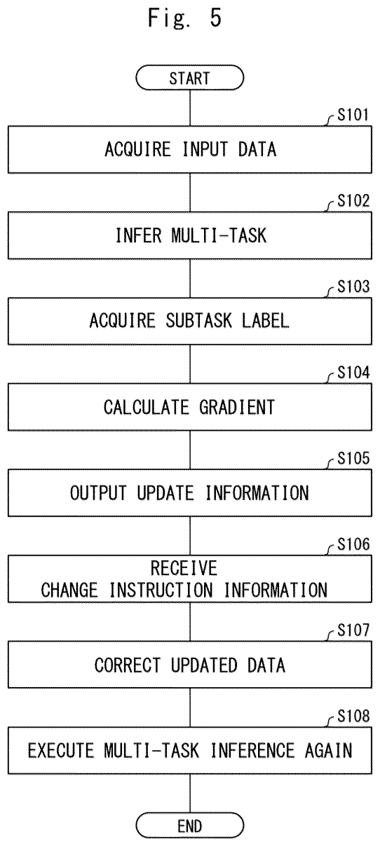

FIG. 5 is a flowchart illustrating an example of a flow of an information processing method according to the present disclosure. In step S101, the data acquisition unit 11A acquires input data. In step S102, the model unit 12A calculates, based on the input data, the first data f, which is a feature, and performs multi-task inference. Furthermore, in step S102, the output control unit 16A outputs the subtask inference result y.

In step S103, the subtask label acquisition unit 13A acquires a part of the subtask labels ^{˜}y from the outside. The number of subtask labels acquired by the subtask label acquisition unit 13A may be one or more. In step S104, the gradient calculation unit 14A calculates the gradient ∇_{f}L_{y} in the first data f, which is the feature, from an error between the subtask label ^{˜}y acquired by the subtask label acquisition unit 13A and the subtask inference result y by the model unit 12A. Furthermore, the re-inference unit 15A updates the first data f using the gradient ∇_{f}L_{y}.

In step S105, the output control unit 16A outputs update information indicating the content of the update by the re-inference unit 15A. The user of the information processing apparatus 1A checks the output update information, and checks whether a portion that should not change has changed. In a case where the user desires to correct the updated data ^{˜}f, such as a case where a portion that should not change has changed, the user inputs change instruction information using an input device or the like.

In step S106, the reception unit 17A receives the change instruction information. In step S107, the re-update unit 18A corrects, based on the change instruction information received by the reception unit 17A, the updated data ^{˜}f obtained by the update of the re-inference unit 15A. In step S108, the re-inference unit 15A executes the multi-task inference again using the re-update data ^{˜˜}f obtained by the correction of the re-update unit 18A and the learned model 21. In the example of FIG. 4, the re-inference unit 15A performs inference by inputting the re-update data ^{˜˜}f instead of the first data f to the second linear layer 213.

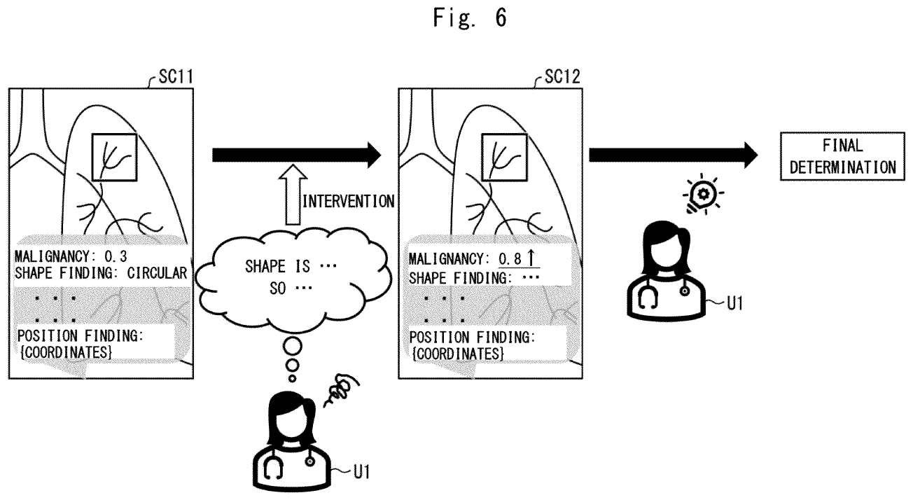

FIG. 6 is a diagram illustrating an example of a flow of task inference using the information processing apparatus 1A. In FIG. 6, a screen SC11 is, for example, a screen displayed on a terminal held by a user U1 of the information processing apparatus 1A, and includes the subtask inference result y output by the output control unit 16A. In the screen SC11, “shape finding: circular”, “position finding: {coordinates}”, and “malignancy: 0.3” each indicate the subtask inference result y.

The user U1 such as a doctor checks the screen displayed on the screen SC11 and inputs the subtask label ^{˜}y. In this manner, the user U1 performs feedback on the finding (the subtask inference result y) output by the learned model 21, and asks for the malignancy diagnosis (subtask 0) by the learned model 21 again. The re-inference unit 15A executes task inference again using the gradient ∇_{f}L_{y} calculated using the input subtask label ^{˜}y.

A screen SC12 is a screen representing a result of the re-inference. The screen SC12 includes subtask inference results such as “shape finding . . . ”, “position finding: {coordinates}”, and “malignancy: 0.8”. By using the gradient ∇_{f}L_{y}, the result of the re-inference is different from the subtask inference result included in the screen SC11. As described above, according to the information processing apparatus 1A, the feedback of the user U1 can be reflected in the inference of the learned model 21. The user U1 checks the result of the re-inference and makes a final determination on the treatment policy and the like.

### Specific Example of Change Information

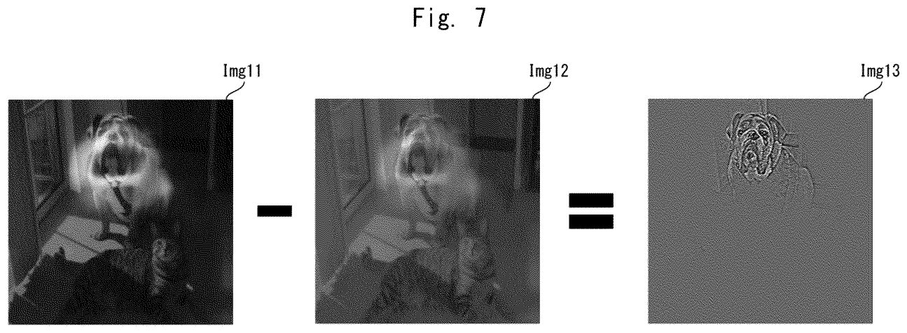

FIG. 7 is a diagram illustrating an example of change information output by the output control unit 16A. In FIG. 7, an image Img11 is an image represented by the first data f before update, and an image Img12 is an image represented by the updated data ^{˜}f. An image Img13 is an image representing a difference between the first data f and the updated data ^{˜}f. By checking these images, the user can grasp how the data has been changed by his/her feedback.

### (Effects of Information Processing Apparatus)

As described above, the information processing apparatus 1A adopts a configuration in which the re-inference unit 15A updates the first data f using the gradient ∇_{f}L_{y} and performs task inference using the updated data ^{˜}f and the learned model 21. In a case where task inference is performed using the subtask label ^{˜}y input by the user, the effect of change by the user can be reflected in the task inference, but the effect is limited. On the other hand, according to the information processing apparatus 1A, not only the subtask inference result y is replaced with the subtask label ^{˜}y, but also the first data f is replaced with the updated data ^{˜}f and is used for inference of other subtasks, so that the feedback by the user on a certain subtask can be effectively reflected in the other subtasks. As described above, according to the information processing apparatus 1A, in a case where the user changes the inference result of any subtask, the effect of the change can be reflected in the inference result of another subtask.

Furthermore, the information processing apparatus 1A adopts a configuration in which the re-inference unit 15A inputs the updated data ^{˜}f to a layer (a third layer) different from the first layer in the task inference. In a case where task inference is performed using the subtask label ^{˜}y input by the user, the effect of change by the user can be reflected in the task inference, but the effect is limited. On the other hand, according to the information processing apparatus 1A, not only the subtask inference result y is replaced with the subtask label ^{˜}y, but also the first data f is replaced with the updated data ^{˜}f and is input to the second linear layer 213 (the third layer), so that the feedback by the user on the subtask can be effectively reflected in the other subtasks.

Furthermore, the information processing apparatus 1A adopts a configuration in which the first data f includes at least one of input data of the learned model 21 and a feature extracted from the input data. By changing at least one of the input data of the learned model 21 and the feature extracted from the input data based on the feedback by the user instead of simply changing the inference result of the subtask and inferring the task using the changed data, the result of the feedback by the user can be effectively reflected in the inference of the task.

Furthermore, the information processing apparatus 1A adopts a configuration including the output control unit 16A that outputs information indicating the content of update by the re-inference unit 15A. For example, the user or the like of the information processing apparatus 1A can grasp how much the feature recognized by the learned model 21 has changed and how the feature has changed by checking the output information.

Furthermore, the information processing apparatus 1A further includes the reception unit 17A that receives change instruction information instructing a change to the updated data ^{˜}f, and the re-update unit 18A that updates the updated data ^{˜}f again using the change instruction information, in which the re-inference unit 15A performs task inference using the re-update data ^{˜˜}f updated by the re-update unit 18A. The user or the like of the information processing apparatus 1A checks the information output by the output control unit 16A, and performs an operation of, for example, in a case where a portion not related to the subtask at all changes, designating the portion not to change. Based on the operation, the re-update unit 18A updates the updated data ^{˜}f and performs task inference using the re-update data ^{˜˜}f, so that task inference that effectively reflects user feedback can be performed.

Furthermore, the information processing apparatus 1A adopts a configuration in which the input data of the learned model 21 is data representing a medical image. Therefore, according to the information processing apparatus 1A, it is possible to obtain an effect that task inference using a medical image as an input can be performed with higher accuracy.

Furthermore, the information processing apparatus 1A adopts a configuration in which the subtask label is a label indicating an inference result of a disease state regarding a medical image. Therefore, according to the information processing apparatus 1A, it is possible to obtain an effect that inference of a disease state from a medical image can be performed with higher accuracy.

### Modified Example

The functions of the information processing apparatus 1 and the information processing apparatus 1A described above may be shared and implemented by a plurality of apparatuses. For example, the information processing apparatus 1 or the information processing apparatus 1A described above may be implemented as an inference system in which two or more apparatuses are connected to each other via a communication network. In this case, as an example, the inference system may be a system including a first device including the data acquisition unit 11A, the subtask label acquisition unit 13A, the output control unit 16A, and the reception unit 17A, and a second device including the model unit 12A, the gradient calculation unit 14A, the re-inference unit 15A, and the re-update unit 18A. In this case, the first device and the second device cooperate with each other to implement the function of the information processing apparatus 1A.

Furthermore, in the above-described information processing apparatus 1A, a case in which the learned model 21 is stored in the storage unit 20A of the information processing apparatus 1A has been described, but the learned model 21 may be stored in a device other than the information processing apparatus 1A. In this case, the information processing apparatus 1A transmits the input data to an apparatus in which the learned model 21 is stored, and receives an inference result of the subtask and an inference result of the task transmitted from the apparatus as a response to the transmitted input data.

Furthermore, in the information processing apparatus 1A described above, data obtained by combining the first data ^{˜}f and the subtask label ^{˜}y is input to the second linear layer 213 (an example of the third layer). The data to be input to the third layer is not limited to data obtained by combining the first data ^{˜}f and the subtask labels ^{˜}y. For example, the first data ^{˜}f obtained by the update may be directly input to the second linear layer 213.

### Example of Implementation by Software

Some or all of the functions of the information processing apparatuses 1 and 1A (hereinafter, also referred to as “each of the above apparatuses”) may be implemented by hardware such as an integrated circuit (an IC chip) or may be implemented by software.

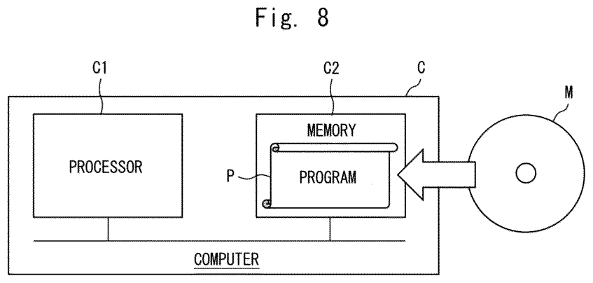

In the latter case, each of the above apparatuses is implemented by, for example, a computer that executes a command of a program which is software for implementing each function. An example of such a computer (hereinafter, referred to as a computer C) is illustrated in FIG. 8. FIG. 8 is a block diagram illustrating a hardware configuration of the computer C functioning as each of the above apparatuses.

The computer C includes at least one processor C1 and at least one memory C2. A program P for causing the computer C to operate as each of the above apparatuses is recorded in the memory C2. In the computer C, the processor C1 reads the program P from the memory C2 and executes the program P to implement each function of each of the above apparatuses.

As the processor C1, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination thereof can be used. As the memory C2, for example, a flash memory, a hard disk drive (HDD), a solid state drive (SSD), or a combination thereof can be used.

Note that the computer C may further include a random access memory (RAM) for loading the program P at the time of execution and temporarily storing various types of data. In addition, the computer C may further include a communication interface for transmitting and receiving data to and from other apparatuses. The computer C may further include an input/output interface for connecting input/output devices such as a keyboard, a mouse, a display, and a printer.

In addition, the program P can be recorded in a non-transitory tangible recording medium M readable by the computer C. As such a recording medium M, for example, a tape, a disk, a card, a semiconductor memory, a programmable logic circuit, or the like can be used. The computer C can acquire the program P via such a recording medium M. In addition, the program P can be transmitted via a transmission medium. As such a transmission medium, for example, a communication network, a broadcast wave, or the like can be used. The computer C can also acquire the program P via such a transmission medium.

The program P can be stored and provided to the computer C using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program P may be provided to the computer C using any type of transitory computer readable media. Examples of transitory computer readable media include electric signals, optical signals, and electromagnetic waves. Transitory computer readable media can provide the program P to the computer C via a wired communication line (e.g. electric wires, and optical fibers) or a wireless communication line.

In addition, each of the above functions of each of the above apparatuses may be implemented by a single processor provided in a single computer, may be implemented by cooperation of a plurality of processors provided in a single computer, or may be implemented by cooperation of a plurality of processors provided in a plurality of computers, respectively. In addition, the program for causing each of the above apparatuses to implement each of the above functions may be stored in a single memory provided in a single computer, may be stored in a distributed manner in a plurality of memories provided in a single computer, or may be stored in a distributed manner in a plurality of memories provided in a plurality of computers, respectively.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example, to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

### Supplementary Notes 1

The whole or part of the example embodiments disclosed above can be described as the following supplementary notes. However, the present disclosure is not limited to the techniques described in the following supplementary notes, and various modifications can be made within the scope described in the claims.

### Supplementary Notes A

The whole or part of the example embodiments disclosed above can be described as the following supplementary notes. However, the present disclosure is not limited to the techniques described in the following supplementary notes, and various modifications can be made within the scope described in the claims.

### Supplementary Note A1

An information processing apparatus including:
a data acquisition means for acquiring data to be input to a first layer included in a plurality of layers forming a learned model generated by multi-task learning;
an inference result acquisition means for acquiring an inference result of a subtask, the inference result being output from a second layer, which is a layer subsequent to the first layer, by inputting the data to the first layer;
a subtask label acquisition means for acquiring a subtask label corresponding to the data;
a gradient calculation means for calculating a gradient in the data of a function using, as inputs, the inference result of the subtask and the subtask label; and
an inference means for performing task inference using the data, the gradient, and the learned model.

### Supplementary Note A2

The information processing apparatus according to supplementary note A1, in which the inference means updates the data using the gradient, and performs the inference using updated data and the learned model.

### Supplementary Note A3

The information processing apparatus according to supplementary note A2, in which the inference means inputs the updated data to a third layer included in the plurality of layers in the inference.

### Supplementary Note A4

The information processing apparatus according to any one of supplementary notes A1 to A3, in which the data includes at least one of input data of the learned model and a feature extracted from the input data.

### Supplementary Note A5

The information processing apparatus according to supplementary note A2 or A3, further including an output control means for outputting information representing a content updated by the inference means.

### Supplementary Note A6

The information processing apparatus according to supplementary note A5, further including:
a reception means for receiving change instruction information instructing a change with respect to the data updated by the inference means; and
a re-update means for re-updating, using the change instruction information, the data updated by the inference means,
in which the inference means performs the inference using data updated by the re-update means.

### Supplementary Note A7

The information processing apparatus according to any one of supplementary notes A1 to A6, in which input data of the learned model is data representing a medical image.

### Supplementary Note A8

The information processing apparatus according to supplementary note A7, in which the subtask label is a label indicating an inference result of a disease state regarding the medical image.

### [Supplementary Notes B]

The whole or part of the example embodiments disclosed above can be described as the following supplementary notes. However, the present disclosure is not limited to the techniques described in the following supplementary notes, and various modifications can be made within the scope described in the claims.

### Supplementary Note B1

An information processing method including:
data acquisition processing of acquiring, by at least one processor, data to be input to a first layer included in a plurality of layers forming a learned model generated by multi-task learning;
inference result acquisition processing of acquiring, by the at least one processor, an inference result of a subtask, the inference result being output from a second layer, which is a layer subsequent to the first layer, by inputting the data to the first layer;
subtask label acquisition processing of acquiring, by the at least one processor, a subtask label corresponding to the data;
gradient calculation processing of calculating, by the at least one processor, a gradient in the data of a function using, as inputs, the inference result of the subtask and the subtask label; and
inference processing of performing, by the at least one processor, task inference using the data, the gradient, and the learned model.

### Supplementary Note B2

The information processing method according to supplementary note B1, in which, in the inference processing, the at least one processor updates the data using the gradient, and performs the inference using updated data and the learned model.

### Supplementary Note B3

The information processing method according to supplementary note B2, in which, in the inference processing, the at least one processor inputs the updated data to a third layer included in the plurality of layers in the inference.

### Supplementary Note B4

The information processing method according to any one of supplementary notes B1 to B3, in which the data includes at least one of input data of the learned model and a feature extracted from the input data.

### Supplementary Note B5

The information processing method according to supplementary note B2 or B3, further including output control processing of outputting, by the at least one processor, information representing a content updated in the inference processing.

### Supplementary Note B6

The information processing method according to supplementary note B5, further including:
reception processing of receiving, by the at least one processor, change instruction information instructing a change with respect to the data updated in the inference processing; and
re-update processing of re-updating, by the at least one processor, the data updated in the inference processing using the change instruction information,
in which, in the inference processing, the at least one processor performs the inference using data updated in the re-update processing.

### Supplementary Note B7

The information processing method according to any one of supplementary notes B1 to B6, in which input data of the learned model is data representing a medical image.

### Supplementary Note B8

The information processing method according to supplementary note B7,
in which the subtask label is a label indicating an inference result of a disease state regarding the medical image.

### Supplementary Notes C

The whole or part of the example embodiments disclosed above can be described as the following supplementary notes. However, the present disclosure is not limited to the techniques described in the following supplementary notes, and various modifications can be made within the scope described in the claims.

### Supplementary Note C1

An information processing program causing a computer to function as an information processing apparatus, the information processing program causing the computer to function as:
a data acquisition means for acquiring data to be input to a first layer included in a plurality of layers forming a learned model generated by multi-task learning;
an inference result acquisition means for acquiring an inference result of a subtask, the inference result being output from a second layer, which is a layer subsequent to the first layer, by inputting the data to the first layer;
a subtask label acquisition means for acquiring a subtask label corresponding to the data;
a gradient calculation means for calculating a gradient in the data of a function using, as inputs, the inference result of the subtask and the subtask label; and
an inference means for performing task inference using the data, the gradient, and the learned model.

### Supplementary Note C2

The information processing program according to supplementary note C1, in which the inference means updates the data using the gradient, and performs the inference using updated data and the learned model.

### Supplementary Note C3

The information processing program according to supplementary note C2, in which the inference means inputs the updated data to a third layer included in the plurality of layers in the inference.

### Supplementary Note C4

The information processing program according to any one of supplementary notes C1 to C3, in which the data includes at least one of input data of the learned model and a feature extracted from the input data.

### Supplementary Note C5

The information processing program according to supplementary note C2 or C3, further causing the computer to function as an output control means for outputting information representing a content updated by the inference means.

### Supplementary Note C6

The information processing program according to supplementary note C5, further causing the computer to function as:
a reception means for receiving change instruction information instructing a change with respect to the data updated by the inference means; and
a re-update means for re-updating, using the change instruction information, the data updated by the inference means,
in which the inference means performs the inference using data updated by the re-update means.

### Supplementary Note C7

The information processing program according to any one of supplementary notes C1 to C6, in which input data of the learned model is data representing a medical image.

### Supplementary Note C8

The information processing program according to supplementary note C7, in which the subtask label is a label indicating an inference result of a disease state regarding the medical image.

### Supplementary Notes D

The whole or part of the example embodiments disclosed above can be described as the following supplementary notes. However, the present disclosure is not limited to the techniques described in the following supplementary notes, and various modifications can be made within the scope described in the claims.

### Supplementary Note D1

An information processing apparatus including at least one processor, in which the at least one processor executes:
data acquisition processing of acquiring data to be input to a first layer included in a plurality of layers forming a learned model generated by multi-task learning;
inference result acquisition processing of acquiring an inference result of a subtask, the inference result being output from a second layer, which is a layer subsequent to the first layer, by inputting the data to the first layer;
subtask label acquisition processing of acquiring a subtask label corresponding to the data;
gradient calculation processing of calculating a gradient in the data of a function using, as inputs, the inference result of the subtask and the subtask label; and
inference processing of performing task inference using the data, the gradient, and the learned model.

Note that the information processing apparatus may further include a memory. In addition, the memory may store a program for causing the at least one processor to execute each processing.

### Supplementary Note D2

The information processing apparatus according to supplementary note D1, in which, in the inference processing, the at least one processor updates the data using the gradient, and performs the inference using updated data and the learned model.

### Supplementary Note D3

The information processing apparatus according to supplementary note D2, in which, in the inference processing, the at least one processor inputs the updated data to a third layer included in the plurality of layers in the inference.

### Supplementary Note D4

The information processing apparatus according to any one of supplementary notes D1 to D3, in which the data includes at least one of input data of the learned model and a feature extracted from the input data.

### Supplementary Note D5

The information processing apparatus according to supplementary note D2 or D3, in which the at least one processor further executes output control processing of outputting information representing a content updated in the inference processing.

### Supplementary Note D6

The information processing apparatus according to supplementary note D5, in which
the at least one processor further executes:
reception processing of receiving change instruction information instructing a change with respect to the data updated in the inference processing; and
re-update processing of re-updating the data updated in the inference processing using the change instruction information, and
in the inference processing, the at least one processor performs the inference using data updated in the re-update processing.

### Supplementary Note D7

The information processing apparatus according to any one of supplementary notes D1 to D6, in which input data of the learned model is data representing a medical image.

### Supplementary Note D8

The information processing apparatus according to supplementary note D7, in which the subtask label is a label indicating an inference result of a disease state regarding the medical image.

### Supplementary Notes E

The whole or part of the example embodiments disclosed above can be described as the following supplementary note. However, the present disclosure is not limited to the techniques described in the following supplementary notes, and various modifications can be made within the scope described in the claims.

### Supplementary Note E1

A non-transitory computer-readable medium storing a program that causes a computer to execute:
data acquisition process of acquiring data to be input to a first layer included in a plurality of layers forming a learned model generated by multi-task learning;
inference result acquisition process of acquiring an inference result of a subtask, the inference result being output from a second layer, which is a layer subsequent to the first layer, by inputting the data to the first layer;
subtask label acquisition process of acquiring a subtask label corresponding to the data;
gradient calculation process of calculating a gradient in the data of a function using, as inputs, the inference result of the subtask and the subtask label; and
inference process of performing task inference using the data, the gradient, and the learned model.

Some or all of elements (e.g., structures and functions) specified in Supplementary Notes A2 to A8 dependent on Supplementary Note A1 may also be dependent on Supplementary Note E1 in dependency similar to that of Supplementary Notes A2 to A8 on Supplementary Note A1. Some or all of elements specified in any of Supplementary Notes may be applied to various types of hardware, software, and recording means for recording software, systems, and methods.

What is claimed is:

1. An information processing apparatus comprising:
at least one memory storing instructions, and
at least one processor configured to execute the instructions to;
acquire data to be input to a first layer included in a plurality of layers forming a learned model generated by multi-task learning;
acquire an inference result of a subtask, the inference result being output from a second layer, which is a layer subsequent to the first layer, by inputting the data to the first layer;
acquire a subtask label corresponding to the data;
calculate a gradient in the data of a function using, as inputs, the inference result of the subtask and the subtask label; and
perform task inference using the data, the gradient, and the learned model.

2. The information processing apparatus according to claim 1, wherein the at least one processor is further configured to execute the instructions to update the data using the gradient, and perform the inference using updated data and the learned model.

3. The information processing apparatus according to claim 2, wherein the at least one processor is further configured to execute the instructions to input the updated data to a third layer included in the plurality of layers in the inference.

4. The information processing apparatus according to claim 1, wherein the data includes at least one of input data of the learned model and a feature extracted from the input data.

5. The information processing apparatus according to claim 2, wherein the at least one processor is further configured to execute the instructions to output information representing a content that is updated.

6. The information processing apparatus according to claim 5, wherein the at least one processor is further configured to execute the instructions to;
receive change instruction information instructing a change with respect to the data updated;
re-update, using the change instruction information, the data that is updated; and
perform the inference using data that is re-updated.

7. The information processing apparatus according to claim 1, wherein input data of the learned model is data representing a medical image.

8. The information processing apparatus according to claim 7, wherein the subtask label is a label indicating an inference result of a disease state regarding the medical image.

9. An information processing method comprising:
data acquisition processing of acquiring, by at least one processor, data to be input to a first layer included in a plurality of layers forming a learned model generated by multi-task learning;
inference result acquisition processing of acquiring, by the at least one processor, an inference result of a subtask, the inference result being output from a second layer, which is a layer subsequent to the first layer, by inputting the data to the first layer;
subtask label acquisition processing of acquiring, by the at least one processor, a subtask label corresponding to the data;
gradient calculation processing of calculating, by the at least one processor, a gradient in the data of a function using, as inputs, the inference result of the subtask and the subtask label; and
inference processing of performing, by the at least one processor, task inference using the data, the gradient, and the learned model.

10. A non-transitory computer-readable medium storing a program that causes a computer to execute:
a data acquisition process of acquiring data to be input to a first layer included in a plurality of layers forming a learned model generated by multi-task learning;
an inference result acquisition process of acquiring an inference result of a subtask, the inference result being output from a second layer, which is a layer subsequent to the first layer, by inputting the data to the first layer;
a subtask label acquisition process of acquiring a subtask label corresponding to the data;
a gradient calculation process of calculating a gradient in the data of a function using, as inputs, the inference result of the subtask and the subtask label; and
an inference process of performing task inference using the data, the gradient, and the learned model.
